(19) United States
(12) Patent Application Publication
(10) Pub. No.: US 2021/0330906 A1
(43) Pub. Date: Oct. 28, 2021

(54) Respiratory Expellant Containment Pad

(71) Applicant: David Ryan SATUR, Fowlerville, MI (US)

(72) Inventor: David Ryan SATUR, Fowlerville, MI (US)

(21) Appl. No.: 16/860,517

(22) Filed: Apr. 28, 2020

(51) Int. Cl. A61M 16/00; A61L 2/26

(52) U.S. Cl. CPC A61M 16/009; A61L 2/26; A61L 2202/16; A61M 2209/088; A61M 2202/203; A61M 2202/206; A61M 2207/10; A61L 2202/26

(57) ABSTRACT

A respiratory expellant containment pad including a base layer and a containment layer on a first side of the base layer. The containment layer is configured to contain respiratory expellant from a wearer of the containment pad. An adhesive layer on a second side of the base layer is opposite to the containment layer.

### FIELD

The present disclosure relates to a respiratory expellant containment pad, which may be secured to a user's arm, sleeve, or cuff, for example.

### BACKGROUND

This section provides background information related to the present disclosure which is not necessarily prior art.

Reducing the spread of germs, such as bacteria, viruses, and other microscopic particles that cause illness, is extremely important, particularly with respect to highly contagious respiratory illnesses, such as severe acute respiratory syndrome coronavirus 2 (SARS-CoV-2). When a person senses that a respiratory expellant is about to occur, such as a sneeze or cough, it is widely recommended that the expellant be directed into the person's arm, such as at the elbow region. While this technique may have some benefits, improvement is possible. The present disclosure provides for an improved way of containing and disinfecting a respiratory expellant, such as germs expelled during a sneeze and/or cough.

### SUMMARY

This section provides a general summary of the disclosure, and is not a comprehensive disclosure of its full scope or all of its features.

The present disclosure includes a respiratory expellant containment pad with a base layer and a containment layer on a first side of the base layer. The containment layer is configured to contain respiratory expellant from a wearer of the containment pad. An adhesive layer is on a second side of the base layer that is opposite to the containment layer.

The present disclosure further includes a respiratory expellant containment pad having a base layer and a containment layer secured to a first side of the base layer. The containment layer includes a disinfectant configured to disinfect respiratory expellant from the wearer of the containment pad including coughing and sneezing. A sidewall extends from the base layer and around the containment layer. An adhesive layer is on a second side of the base layer that is opposite to the containment layer. A predefined crease extends across a width of the pad. The predefined crease is configured to facilitate bending of the pad, such as when adhered at a user's elbow region.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 illustrates an exemplary respiratory expellant containment pad in accordance with the present disclosure adhered to a user's arm;

FIG. 2 is a perspective view of the respiratory expellant containment pad of FIG. 1;

FIG. 3 is a cross-sectional view taken along line 3-3 of FIG. 2;

FIG. 4 is a cross-sectional view of the respiratory expellant containment pad of FIG. 1 showing peelable adhesive protective strips being removed therefrom; and

FIG. 5 is a cross-sectional view of the respiratory expellant containment pad of FIG. 1 adhered to a user's arm at the user's elbow.

Corresponding reference numerals indicate corresponding parts throughout the several views of the drawings.

### DETAILED DESCRIPTION

Example embodiments will now be described more fully with reference to the accompanying drawings.

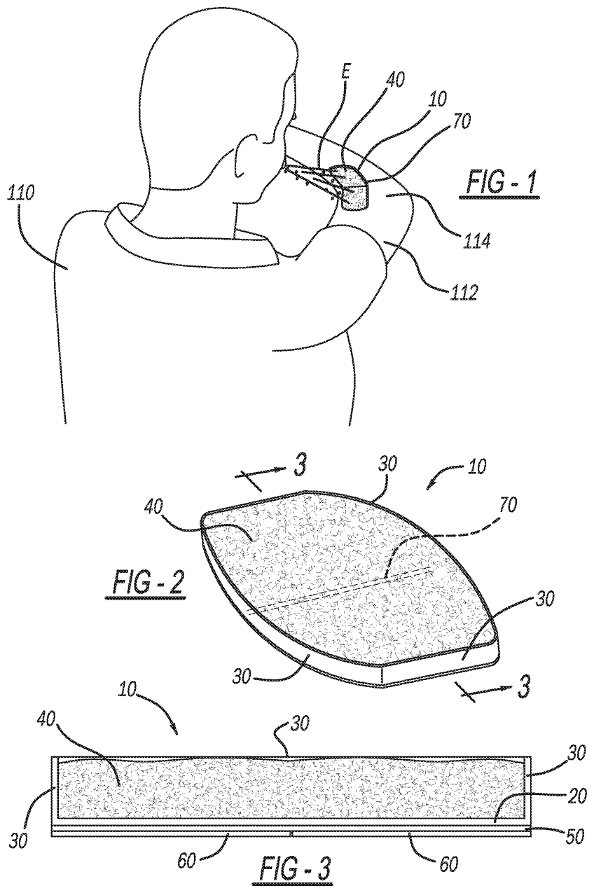

FIG. 1 illustrates an exemplary respiratory expellant containment pad 10 in accordance with the present disclosure adhered to a person or user 110 to absorb and/or disinfect respiratory expellant E from the user 110. The containment pad 10 may be adhered to any area of the user 110, such as the user's arm 112, and particularly at the user's elbow 114. The containment pad 10 may be adhered to any other area of the user 110 as well, or to any portion of the user's clothing. The containment pad 10 is typically arranged at a location where it is most convenient for the user 110 to direct expellant E to the containment pad 10.

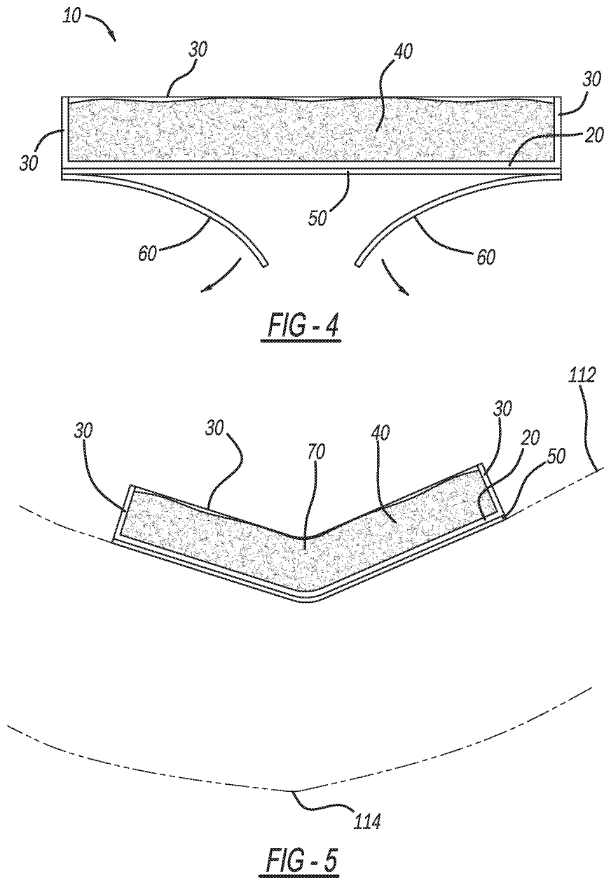

With continued reference to FIG. 1, and additional reference to FIGS. 2-5, the containment pad 10 will be described in additional detail. The containment pad 10 generally includes a base layer 20, a sidewall 30, a containment layer 40, an adhesive layer 50, and adhesive protective strips 60.

The base 20 may be made of any suitable material, such as any suitable polymeric material. The base 20 may be made of an impervious plastic sheathing, for example. The base 20 may be of any suitable size. For example, the base 20 may have any length and width suitable for being secured to the arm 112 of the person 110, such as at or near the elbow 114. The base 20 may have a width of 4 inches, or about 4 inches, and a length of six inches, or about six inches.

The sidewall 30 extends from the base 20. The sidewall 30 may be integral or monolithic with the base 20. Alternatively, the sidewall 30 may be secured to the base 20 in any suitable manner. The sidewall 30 may extend from the base 20 a right angle, for example, or at any other suitable angle relative to the base 20. The sidewall 30 extends around at least a portion of the containment layer 40, and may extend entirely around the containment layer 40. The sidewall 30 may extend further from the base 20 than the containment layer 40. Thus, the sidewall 30 is configured to prevent or reduce spill-over of the expellant E, and thus the sidewall 30 helps confine the expellant E to the pad 10 and the containment layer 40.

The containment layer 40 is made of any material, natural or synthetic, configured to contain the respiratory expellant E at the pad 10, such as coughing and sneezing. The containment layer 40 may be a woven or non-woven material. The containment layer 40 may include a disinfectant, such as any disinfectant suitable for disinfecting the respiratory expellant E from the person 110. The containment layer 40 may be secured to the base 20 in any suitable manner, such as with any suitable adhesive. Materials that may be included in the containment layer 40 include, but are not limited to, any suitable alcohol-based sanitizer or disinfectant. The containment layer 40 may include any material having silver ion SDC technology, such as PureGreen24 available from Pure Green LLC.

On a side of the base 20 opposite to the containment layer 40 is an adhesive layer 50. The adhesive layer 50 includes any adhesive suitable to secure the pad 10 to the skin of the user 110, such as at the arm 112 and particularly at the elbow 114. The adhesive of the adhesive layer 50 may also be suitable to, or alternatively be suitable to, secure the pad 10 to clothing of the user 110. The adhesive layer 50 is covered by peelable adhesive protective strips 60, which are peeled off when the user 110 is ready to attached the pad to his/her skin or clothing.

The pad 10 may include a crease 70 to facilitate bending of the pad 10. The crease 70 may extend across a width of the pad 10, for example. The crease 70 may be defined by at least one of the containment layer 40, the base 20, and the adhesive layer 50. The pad 10 may be arranged along the arm 112 at the inner elbow 114, with the crease 70 where the arm 112 bends. The crease 70 thus makes it easier for the person 110 to bend his/her elbow.

The pad 10 provides numerous advantages. For example, when the user 110 feels that a cough, sneeze, or other respiratory expellant E is about to occur, he/she may position the pad 10 at his/her mouth and/or nose to direct the respiratory expellant E into the pad 10, which absorbs the respiratory expellant E and disinfects it. The sidewall 30 helps to contain the respiratory expellant E at the containment layer 40 because the sidewall 30 extends above and around the containment layer 40. The pad 10 may be easily removed and replaced by another unused pad after the pad 10 has been used a number of times to absorb and disinfect expellant E. One skilled in the art will appreciate that the present disclosure provides numerous additional advantages as well.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A respiratory expellant containment pad comprising:
a base layer;
a containment layer on a first side of the base layer, the containment layer configured to contain respiratory expellant from a wearer of the containment pad; and
an adhesive layer on a second side of the base layer that is opposite to the containment layer.

2. The respiratory expellant containment pad of claim 1, wherein the containment layer includes a disinfectant and is configured to disinfect respiratory expellant from the wearer of the containment pad.

3. The respiratory expellant containment pad of claim 1, wherein the containment layer is woven.

4. The respiratory expellant containment pad of claim 1, wherein the containment layer is non-woven.

5. The respiratory expellant containment pad of claim 1, wherein the containment layer is a natural material.

6. The respiratory expellant containment pad of claim 1, wherein the containment layer is synthetic.

7. The respiratory expellant containment pad of claim 1, wherein containment layer is configured to contain respiratory expellant including coughing and sneezing.

8. The respiratory expellant containment pad of claim 1, wherein the base layer is about 4 inches wide and six inches long.

9. The respiratory expellant containment pad of claim 1, wherein the base layer includes impervious plastic sheathing.

10. The respiratory expellant containment pad of claim 1, wherein the containment layer is secured to the base layer.

11. The respiratory expellant containment pad of claim 1, further comprising a sidewall extending about the containment layer.

12. The respiratory expellant containment pad of claim 11, wherein the sidewall extends completely around the containment layer.

13. The respiratory expellant containment pad of claim 12, wherein the sidewall extends further from the base layer than the containment layer.

14. The respiratory expellant containment pad of claim 1, further comprising at least one peelable protective strip adhered to the adhesive layer, the peelable protective strip is removable to expose the adhesive layer, the adhesive layer configured to secure the containment pad to skin or fabric.

15. The respiratory expellant containment pad of claim 1, further comprising a predefined crease extending across a width of the pad, the predefined crease configured to facilitate bending of the pad.

16. A respiratory expellant containment pad comprising:
a base layer;
a containment layer secured to a first side of the base layer, the containment layer including a disinfectant configured to disinfect respiratory expellant from the wearer of the containment pad including coughing and sneezing;
a sidewall extending from the base layer and extending around the containment layer;
an adhesive layer on a second side of the base layer that is opposite to the containment layer; and
a predefined crease extending across a width of the pad, the predefined crease configured to facilitate bending of the pad.

17. The respiratory expellant containment pad of claim 16, wherein the sidewall extends completely around the containment layer.

18. The respiratory expellant containment pad of claim 17, wherein the sidewall extends further from the base layer than the containment layer.
